(19) United States
(12) Patent Application Publication
(10) Pub. No.: US 2009/0183753 A1
(43) Pub. Date: Jul. 23, 2009

(54) METHOD FOR EVALUATION AND GUARANTEEING THERMAL HYGIENIC EFFECTS

(76) Inventors: Bruno Maennle, Durbach (DE); Bruno Gaus, Offenburg (DE); Denis Lehmann, Ortenberg (DE)

Correspondence Address: Muncy, Geissler, Olds & Lowe, PLLC, P.O. BOX 1364, FAIRFAX, VA, 22038-1364, US

(21) Appl. No.: 11/719,746

(22) PCT Filed: Oct. 31, 2005
(86) PCT No.: PCT/EP05/11661

§ 371 Date: Oct. 3, 2008

(30) Foreign Application Priority Data
Nov. 19, 2004 (DE) 10 2004 056 052.8

(51) Int. Cl. A47L 15/42; A47L 15/46; A61L 2/24; A47L 15/00

(52) U.S. Cl. 134/18; 134/113

(57) ABSTRACT

The invention relates to a method for assessing and guaranteeing the thermal hygiene effect on items to be washed which are accommodated in a washing chamber of a single-chamber dishwasher or items to be cleaned which are accommodated in a cleaning chamber of a cleaning and disinfecting machine, with a temperature sensor being permanently assigned to the washing chamber or the cleaning chamber. The thermal hygiene effect produced by the temperature and action time acting on the items to be washed or the items to be cleaned is detected by means of the temperature sensor and a control system, and the heat equivalent values are calculated from this information.

### TECHNICAL FIELD

The present invention relates to a method for assessing and guaranteeing the thermal hygiene effect, in particular of a dishwasher, on the items to be cleaned during the program run in the dishwasher.

### PRIOR ART

EP 1 371 319 B1 discloses a dishwasher having a washing container which can be divided into two parts. A dishwashing apparatus comprises an electrically operated valve which controls the entry of water from a mains, and also a softener unit for water, and also a washing container, a discharge line and a microprocessor. The washing container comprises two frames which are arranged side by side and also a removable central partition wall which divides the container into two independent sectors, of which each is equipped with a collecting trough with a suitable filter, from which collecting trough the water flows to a flushing pump and a discharge pump. A heating resistor, a detergent distributor and at least one sprinkler apparatus are also provided. Water is supplied from the softener unit via a deviation valve which conducts the water to the respective supply lines of sectors. Water is discharged through a connector which combines the discharge lines of the two sectors to form one discharge line and by virtue of a four-way distributor being provided, with the distributor receiving water from the flushing pumps through corresponding lines. The flow in these lines is controlled by respective valves, with the distributor likewise connecting the two troughs to one another by corresponding lines which are separated by means of a central valve of the distributor.

EP 1 374 754 A1 discloses a fitted dishwasher with an increased load capacity. A loading door of the fitted dishwasher is formed by a front panel which is surrounded by a frame. The frame has an increased height and is provided with an abutment area which is deep-drawn and runs within a recess, with the result that space is saved.

DE 101 56 559 C2 discloses a device for temperature measurement control in the washing container of a dishwasher. The dishwasher comprises one or more inserts for items to be washed or dish racks, having a washing container which can be closed by a door. The washing container is provided with a separate probe duct, which is directed out of the appliance, for measurement-probe lines which can be inserted into the container. The probe duct preferably issues into the washing container on the upper face, preferably starting from the front face of the dishwasher. The probe duct which issues into the washing container from the upper face is provided with a measurement opening which is arranged in the upper appliance region such that it is accessible, with the measurement opening being provided close beneath the appliance cover in the region of the appliance door. The measurement opening can also be formed in the region of the upper edge of the appliance door of the washing container. The probe duct is also formed as an angled passage duct for the measurement-probe lines and can be closed at least at one end.

A portion of the probe duct which issues at an angle into the washing container on its upper face can be screwed by means of a threaded stop; the probe duct can also be fixed, preferably releasably, in the ceiling wall part of the washing container. The probe duct is formed in an inclined manner in the appliance with an incline toward the washing space. The cross section of the probe duct is dimensioned such that a plurality of measurement-probe lines can be inserted into the washing container through one and the same probe duct for the purpose of temperature measurement control in the washing container.

In program-controlled dishwashers, in particular in washer machines for laboratories and hospitals which operate with temperature-dependent programs, for example disinfection programs or the like, and serve to treat medical items to be washed, there is a legal requirement for each washer machine, for example a thermal disinfector, to be monitored at regular time intervals to check whether the required disinfection temperature is reached. To this end, temperature measurements have to be carried out at several points in the washing space using a measurement system. In this case, up to 5 sensors, depending on requirements, are fitted in the washing space through a special measurement opening. The probes, which are formed as NTC sensors and/or PT100 elements for example, comprise measurement-probe lines composed of uninsulated wires with a diameter of approximately 0.5 mm and a length of approximately 1 m, or insulated cables. It is known to lay the measurement-probe lines through the door gap between appliance door and washing-space seal or door seal in order to carry out monitoring measurements. However, laying lines in this way is problematic in the case of relatively thick sensor cables. Since temperature-measurement control within the washing container can be carried out only when the appliance door is closed, parts of the door seal are so severely deformed by the clamped lines that leakages occur. A separate measurement opening in the appliance door would have the disadvantage that, when the door is closed, the free cables move in an uncontrolled manner in the washing space and may even reach the rotary region of the spray arms, which are associated with a rack, and block said spray arms.

The dishwashing machines known from the prior art usually have at least one washing chamber and a washwater tank. A circulation pump sucks up washwater from the washwater tank and sprays it onto the dirty items to be cleaned within the washing chamber by means of a washing system. The sprayed washwater then falls back into the washwater tank and is again sucked up by the circulation pump and sprayed onto the items being washed.

In dishwashers which are normally used in the commercial sector, after the items to be washed have been precleaned by the washwater from the washwater tank, either hot post-washwater or fresh water is sprayed over the items to be cleaned by means of a further spray system in a further program step or the dirty washwater is replaced by hot fresh water and sprayed over the items to be cleaned by the same system. The microbial reduction which is achieved in dishwashers after the program run depends to a considerable extent on the temperature of the washwater or post-washwater, the washing duration, the mechanical washing effect and also the concentration of cleaner.

In order to assess the hygiene of the products being washed after the washing program run, test methods are used which are based on microbial reduction in specific test microbes on the surface of the products which have been washed.

DIN 10511-12 C.3 for Germany describes a method in which microbial reduction on specifically contaminated glasses is determined after the program run by means of so-called swab tests. In this case, it is necessary to achieve a minimum degree of microbial reduction in the shortest and/or an unsuitable washing program. The test microbe or organism used in this test is E. faecium ATCC 6057. The results of this test then provide minimum requirements for temperature, washing duration, cleaner concentration and mechanical washing powers, with which this washer or this series of washers then has to be preset by the manufacturer in order to provide the required microbial reduction during operation on the customer's premises. One disadvantage of this method is the fact that these test methods can only be carried out on the customer's premises with an extremely great deal of outlay and are therefore not suitable for checking for correct operation of the washer in the case of small washers.

A method which is described by the NSF standard 3 is known from the USA. The basis for this method is the reduction of tuberculosis bacteria determined in trials by virtue of the effect of a temperature over time. The effect of temperature over time is called the “heat equivalent” (HUE). The number of heat equivalents reached per second at a particular temperature is set out in a table which forms the basis of this method. Trials have been used for washers to define a minimum tank temperature and a minimum post-wash temperature which the washer has to reach in order to achieve the required microbial reduction. For manufacturers of washers, this means that these temperatures have to be permanently preset in the control system of the dishwasher by the manufacturer. When testing the operation of a dishwasher according to this method, a temperature sensor is arranged on a plate. This plate is then placed in a predefined position in the dish rack of the dishwasher and the temperature during the program run is recorded. The heat equivalents acting on the plate throughout the entire program run are then determined from the temperature profile during the entire washing program with reference to the abovementioned table. This test has to be carried out for three different plate positions within the dish rack. In order to achieve the required microbial reduction, regulation NSF-3 states that at least 3600 heat equivalents have to be reached in each plate position within the dish rack. One advantage of this method is that it can be carried out at relatively little expense at the customer's premises in order to check that the washer is operating correctly.

In Europe, prEN ISO 15883-1, which also draws on the connection between the microbial reduction and the temperature over time in order to assess hygiene efficiency, applies to cleaning/disinfecting appliances. This connection is called the A₀ value and is likewise set out in table form or calculated using a mathematical formula. The A₀ value is defined as the time equivalent in seconds at a temperature of 80° C., at which a given disinfecting effect is exerted, and corresponds analogously to the heat equivalents (HUE) of the NSF standard 3. A minimum A₀ value to be reached at each point in the washing chamber of the cleaning and disinfecting appliance has to be guaranteed here too. This accordingly refers to the most unsuitable point in the most unsuitable program. However, this method is not currently used for assessing the hygiene effect of commercial washers in Europe.

All of the methods outlined above share the common feature that the most unsuitable washing program and/or the most unsuitable conditions for assessing microbial reduction are always used. However, one disadvantage of this procedure is that unnecessarily long program run times sometimes result on account of the fixed prespecifications for washing programs whose temperature and other program parameters differ from these prespecifications, and this is extremely undesirable. Therefore, the program run time could be shortened, with the degree of microbial reduction remaining the same, by increasing the washing and/or post-wash temperature for example.

In view of the abovementioned disadvantages of the methods which have been used to date to determine the hygiene effect in dishwashing machines, the present invention is based on the object of providing a method which avoids the disadvantages of the above methods known from the prior art and at the same time permits process reliability in terms of guaranteeing and permanently ensuring the thermal hygiene effect within a dishwashing machine.

### DISCLOSURE OF THE INVENTION

The invention proposes providing a temperature sensor in the washing chamber of the washer, which temperature sensor detects the temperature prevailing in the washing chamber during the entire program run of the dishwashing machine. The temperature sensor is a fixed constituent part of the dishwasher and is accordingly permanently installed in the dishwasher. The temperature sensor is calibrated with the temperature sensor employed in an NSF standard 3 test or in the A₀ test in such a way that it is possible to detect the same heat equivalents as in the NSF-3 standard test method or in the A₀ test method using a control system which is arranged inside or outside the dishwasher. The temperature sensor which is arranged inside the washing chamber of the dishwasher and the control system which is associated with said temperature sensor can be used to determine the instantaneous heat equivalents within the washing chamber of the dishwashing machine. The table values of the NSF-3 standard or the A₀ test method can be stored in the control system, which is provided within the dishwashing machine or on the outside of the dishwashing machine and to which the temperature sensor is connected, and are compared with the values determined by means of the temperature sensors which are arranged within the washing chamber.

If a value, which is preset in the control system of the dishwasher or an external control system which is provided on the outside, for the heat equivalents is reached, an optical or an acoustic signal, for example, can be used to indicate the end of the program run. This is then terminated, with the result that an extremely undesirable extension of the program run time can be avoided. This saves resources and considerably reduces power consumption by the dishwashing machine during operation in each program run.

In one advantageous refinement of the idea which forms the basis of the invention, the end of the program run can be coupled, for example, with unlocking of the appliance door of the dishwasher. After the respectively required heat equivalents which are detected by means of the temperature sensors are reached, the locking mechanism of the appliance door can be activated and the appliance door unlocked. This ensures that the items to be cleaned and possibly disinfected cannot be removed from the dishwasher before the required heat equivalents are reached within the washing chamber.

On account of the method proposed according to the invention, the washing program can, in the case of program runs in the “washing” and/or “post-washing” program step, be terminated in good time after the preset heat equivalents which are preset by the internally or externally provided control system of the temperature sensor are reached and under the condition that the post-washing step is executed, and a superfluous further extension in program run time is prevented.

A further advantage of the solution proposed according to the invention can be seen in that the dishwasher actively monitors the hygiene, that is to say the degree of disinfection of the items to be cleaned, and can automatically correct irregularities in the washing operation, for example the introduction of a larger amount of cold water, using suitable measures. A suitable measure which can be taken when a larger amount of cold water is introduced into the washing chamber is, for example, extending the run time of the post-washing step until signaling of the program end is indicated either optically or acoustically or else by unlocking the appliance door.

The method proposed according to the invention, in which the values for the heat equivalents to be achieved in accordance with the NSF3 standard and in accordance with A₀ value methods are implemented in a control system which is provided externally or internally on the dishwasher, ensures that a thermal hygiene effect which corresponds to the prespecifications is always achieved under the optimum and/or shortest program run time. By virtue of the method proposed according to the invention, in which the values for the equivalents are implemented in the control system, the heat equivalent values which are achieved during the program run can be indicated by means of a display which is actuated by the control system. As a result, the operator is able to read off the respective level of the heat equivalents achieved for the thermal hygiene effect at any time during the program run and to assess progress of the program.

Cleaning and disinfection machines (bedpan washers) which are used in a hospital or care home and in which bedpans, urine bottles and other collection containers for human excreta are cleaned and disinfected can also be operated by virtue of the method proposed according to the invention. Disinfection of these containers can be performed both thermally, that is to say using steam, and also chemically. During operation of such cleaning and disinfecting machines which disinfect using steam, the method proposed according to the invention permits the thermal hygiene effect of said steam on the containers to be cleaned to be assessed. To this end, the cleaning chamber of these machines is likewise provided with a temperature sensor which detects the temperature prevailing in the cleaning chamber during the entire program run within the cleaning and disinfecting machine. The temperature sensor, which is provided in the cleaning chamber of the cleaning and disinfecting machine, is calibrated with a temperature sensor which is used in an NSF standard 3 test or in an A₀ test, with the result that the temperature reached at a particular moment and action time on the items to be cleaned is detected by means of the control system of the cleaning and disinfecting machine and the corresponding heat equivalents can be calculated from this information.

### DRAWING

The method proposed according to the invention is explained in greater detail below on the basis of a single-chamber dishwashing machine and on the basis of a cleaning and disinfecting machine with reference to the drawing, in which:

FIG. 1 shows a schematic reproduction of a single-chamber dishwasher, and

FIG. 2 shows a schematic illustration of a cleaning and disinfecting machine.

### DESIGN VARIANTS

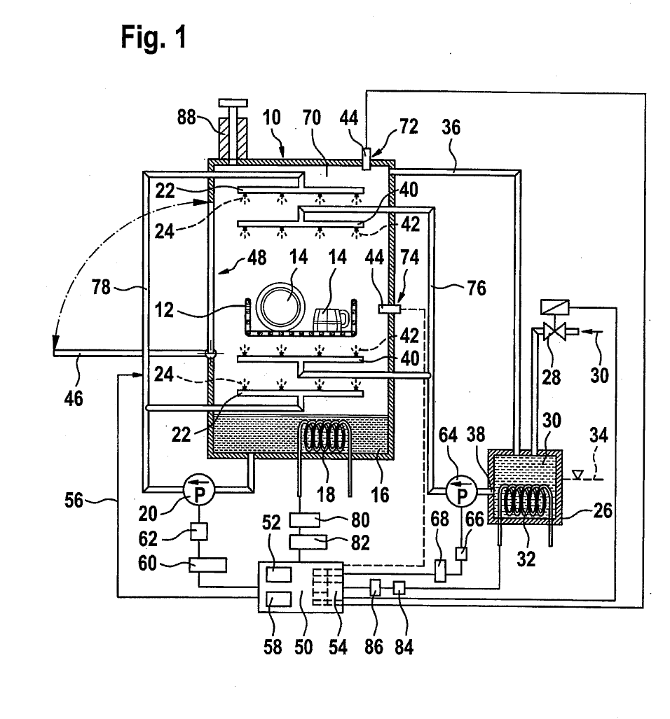

FIG. 1 shows a dishwasher which is a single-chamber dishwasher which can be loaded with items to be cleaned via an appliance door.

A single-chamber washer 10 comprises a washing chamber 70 which accommodates a rack 12. The rack 12 contains items 14 to be washed which may be plates, cups, cutlery, glasses, pots and pans etc. The lower region of the single-chamber washer 10 contains a tank 16 for holding the washing liquor to which cleaner has been added. The washing liquor, which is contained in the tank 16, is heated by means of a heating element 18. The washing liquor is transported from the tank 16 of the single-chamber washer 10 to a washing system 22 for washing liquor by means of a circulation pump 20. The washing system 22 for washing liquor is connected to the circulation pump 20 via a washing-liquor line 78. The washing system 22 for washing liquor, which washing system preferably comprises rotary arms having nozzles 24, is arranged both in the upper region of the washing chamber 70 and in the lower region thereof.

The single-chamber dishwasher 10 is also assigned a fresh-water boiler 26. A filling valve 28 is connected upstream of the fresh-water boiler 26 and the inflow of fresh water 30 into the fresh-water boiler 26 can be controlled by means of said filling valve. The filling valve 28 is connected to a control system 50 of the single-chamber dishwasher, which control system, for its part, controls the program run of the individual program steps running in the single-chamber dishwasher 10. The fresh-water boiler 26 contains a heating element 32 with which the fresh water 30 which subsequently flows into the fresh-water boiler 26 and the fresh water 30 located in the fresh-water boiler 26 are heated. The level of fresh water 30 within the fresh-water boiler 26 is denoted by reference symbol 34 and is always such that the heating element 32 within the fresh-water boiler 26 is covered by fresh water 30. The fresh-water boiler 26 and the washing chamber 70 of the single-chamber dishwasher 10 are connected via a ventilation line 36. A suction point 38 via which a fresh-water pump 64 conveys heated fresh water 30 to washing systems 40 for fresh water via a fresh-water line 76 is provided on the fresh-water boiler 26. The washing systems 40 for fresh water are likewise preferably rotary arms whose sides which face the rack 12 are fitted with nozzles 42 by means of which the heated fresh water 30 is discharged onto the items 14 to be cleaned.

The washing chamber 70 of the single-chamber dishwasher 10 contains a temperature sensor 44 which can be arranged either in a first temperature-sensor position 72 on the ceiling of the washing chamber 70 or can be held in a second temperature-sensor position 74 on the rear wall of the washing chamber 70 of the single-chamber dishwasher 10. The installation position 72, 74 of the temperature sensor 44 is illustrated by way of example; the position of the temperature sensor 44 is, depending on the other boundary conditions, selected such that the determined temperature at the temperature sensor 44 during the program run is comparable with the temperature profile of the items to be washed or of the items to be cleaned.

The temperature sensor 44 is connected to the control system 50 of the single-chamber dishwasher 10. The washing chamber 70 of the single-chamber dishwasher 10 is accessible via an appliance door 46 which opens or closes a loading opening 48. The appliance door 46 is assigned a locking mechanism by means of which the appliance door 46 is locked in its closed position. The locking mechanism for the appliance door 46 is likewise connected to the control system 50 of the single-chamber dishwasher 10.

The control system 50, which is assigned to the single-chamber dishwasher 10, can be either an internal control system, that is to say a control system which is arranged within the single-chamber dishwasher 10, or else an external control system, that is to say a control system provided outside the single-chamber dishwasher 10. The control system 50 comprises a microprocessor (CPU) 52 and also a data memory 54. All the functions in terms of the program steps running in the single-chamber dishwasher 10, that is to say including the method running within the single-chamber dishwasher 10 for assessing the hygiene effect, are controlled via a main control line 56. The control system 50 also comprises a measurement-data detection unit 58 by means of which the measurement values detected by the at least one temperature sensor 44, which is assigned to the washing chamber 70, are stored.

The control system 50 also uses a power controller 60 which is connected upstream of the circulation pump 20 to control the electrical power supply 62 of said circulation pump. A power controller 68 by means of which the electrical power supply 66 of the fresh-water pump 64 can be controlled is also connected upstream of the fresh-water pump 64. The same applies to a power controller 82 by means of which the power supply 80 of the heating element 18 for the washing liquor is connected upstream, and to a power controller 86 which controls the power supply 84 of the heating element 32 of the fresh-water boiler 26.

The values for the heat equivalents, which are applicable either in accordance with the NSF3 standard or in accordance with the A₀ value method, are stored in the control system 50 within the data memory 54 provided there, in order to determine or classify the hygiene effect of a dishwashing machine. The data memory 54 of the control system 50 can store, for example, the A₀ values given below in accordance with EN-ISO 15883 which is to be expected in Europe:

| Tem- | | | A₀3000 | |
|---|---|---|---|---|
| perature | A₀60 (sec) | A₀600 (sec) | (sec) | =(min) |

| | | | | |
|---|---|---|---|---|
| 65° | 1897.4 | 18 973.7 | 94 863.3 | 1581.1 |
| 66° | 1507.1 | 15 071.3 | 75 356.6 | 1255.9 |
| 67° | 1197.2 | 11 971.6 | 59 857.9 | 997.6 |
| 68° | 950.9 | 9509.4 | 47 546.8 | 792.4 |
| 69° | 755.4 | 7553.6 | 37 767.8 | 629.5 |
| 70° | 600.0 | 6000.0 | 30 000.0 | 500.0 |
| 71° | 476.6 | 4766.0 | 23 829.8 | 397.2 |
| 72° | 378.6 | 3785.7 | 18 928.7 | 315.5 |
| 73° | 300.7 | 3007.1 | 15 035.6 | 250.6 |
| 74° | 238.9 | 2388.6 | 11 943.2 | 199.1 |
| 75° | 189.7 | 1897.4 | 9486.8 | 158.1 |
| 76° | 150.7 | 1507.1 | 7535.7 | 125.6 |
| 77° | 119.7 | 1197.2 | 5985.8 | 99.8 |
| 78° | 95.1 | 950.9 | 4754.7 | 79.2 |
| 79° | 75.5 | 755.4 | 3776.8 | 62.9 |
| 80° | 60.0 | 600.0 | 3000.0 | 50.0 |
| 81° | 47.7 | 476.6 | 2383.0 | 39.7 |
| 82° | 37.9 | 378.6 | 1892.9 | 31.5 |
| 83° | 30.1 | 300.7 | 1503.6 | 25.1 |
| 84° | 23.9 | 238.9 | 1194.3 | 19.9 |
| 85° | 19.0 | 189.7 | 948.7 | 15.8 |
| 86° | 15.1 | 150.7 | 753.6 | 12.6 |
| 87° | 12.0 | 119.7 | 598.6 | 10.0 |
| 88° | 9.5 | 95.1 | 475.5 | 7.9 |
| 89° | 7.6 | 75.5 | 377.7 | 6.3 |
| 90° | 6.0 | 60.0 | 300.0 | 5.0 |
| 91° | 4.8 | 47.7 | 238.3 | 4.0 |
| 92° | 3.8 | 37.9 | 189.3 | 3.2 |
| 93° | 3.0 | 30.1 | 150.4 | 2.5 |
| 94° | 2.4 | 23.9 | 119.4 | 2.0 |
| 95° | 1.9 | 19.0 | 94.9 | 1.6 |

The A₀ value that can be taken from the above table is defined as the time equivalent in seconds, at which a disinfection action is exerted.

The A₀ value of a disinfection process with moist heat characterizes the killing of germs, given as the time equivalent in seconds at a temperature transmitted to the product by the process.

The temperature sensor 44 which is used in the washing chamber 70 of the single-chamber dishwasher 10, be it in the first temperature-sensor position 72 or alternatively in the second temperature-sensor position 74, is calibrated to the temperature sensor used in the NSF3-standard test method or within the A₀ test method in such a way that the same heat equivalents as in the NSF3-standard method or in the A₀ test method can be determined by means of the control system 50 within the single-chamber dishwasher 10. The heat equivalents reached at a particular moment within a program run are determined by means of the temperature sensor 44 provided within the washing chamber 70 and the control system 50 and are compared with the table values stored in the data memory 54, for example the values stored there in the case of EN ISO 15883. If the values determined by means of the temperature sensor 44 for the heat equivalents which prevail within the washing chamber 70 of the single-chamber dishwasher 10 are too low, either the temperature of the washing liquor which is present in the washing-liquor tank 16 can be increased by means of the control system 50, or the temperature of the fresh water 30, which is supplied to the washing chamber 70 by means of the washing systems 40, can be increased by means of the control system 50. To this end, the corresponding power controllers 60 and 68, which are assigned to the circulation pump 20 and, respectively, the fresh-water pump 64, are actuated by means of the control system 50.

If the values predefined in accordance with the NFS3 standard or in accordance with EN ISO 15883 for the heat equivalents input to the washing chamber 70 of the single-chamber dishwasher 10 are reached, the program step running in the washing chamber 70 of the single-chamber dishwasher 10 is terminated. The end of the respective program step running in the washing chamber 70 can be indicated either optically, for example by lighting up the display, or else acoustically by the control system 50. It is also possible to unlock the locking mechanism, which is assigned to the appliance door 46 of the single-chamber dishwasher 10, by means of the control system 50 after the prescribed heat equivalents are reached within the washing chamber 70. In this case, it is ensured that the items 14 to be washed are not removed from the washing chamber 70 before the required heat equivalents are reached, that is to say removal from the washing chamber 70 of items 14 to be washed which have been only inadequately thermally treated can be prevented.

By virtue of the method proposed according to the invention for assessing the hygiene effect of the single-chamber dishwasher 10, the respective washing programs can, in the case of program runs with an elevated washing-liquid temperature in the “washing” and/or “post-washing” program step, be terminated after the preset values for the heat equivalents are reached, with the condition that the post-washing step has already been executed.

A further advantage which can be achieved with the method for assessing the hygiene effect, which is proposed according to the invention and implemented in a single-chamber dishwasher 10, can be seen in that the dishwasher actively monitors the hygiene of the items 14 to be washed being cleaned. Irregularities in the washing operation, for example the introduction of a large amount of cold water into the tank for washing liquor 16 and a resulting drop in temperature in the washing chamber 70, can be compensated for by suitable countermeasures initiated by means of the control system 50. Therefore, the control system 50 can, for example, extend the post-washing process, or the temperature of the washing liquor can be correspondingly increased by means of corresponding actuation of the power controller 82 for the heating element 18 of the tank for washing liquor 16, in order to counteract the influx of cold water and prevent a drop in the temperature prevailing in the washing chamber 70 of the single-chamber dishwasher 10.

By virtue of the method proposed according to the invention, it is now possible to detect the respective heat equivalents in each program run independently of irregularities, for example caused by items being washed having different weights or temperatures before the start of the washing program, and to evaluate said heat equivalents in accordance with the prespecifications from the NSF 3 regulation or the EN ISO 15883-1 standard, and to control the program run in an optimum manner. The heat equivalent values reached can be indicated by means of a display. The user of the appliance is therefore provided with the option of tracking or monitoring the thermal hygiene effect of a single-chamber dishwashing machine during each program run.

Reference numeral 88 identifies a locking device which can be formed, for example, as an electromagnet and which can be coupled to the control system 50. After the prescribed heat equivalents are reached within the washing chamber 70 of the single-chamber dishwasher 10, this locking device 88 is unlocked by means of the control system 50. This ensures that only items 14 to be washed which have been exposed to the heat equivalents prescribed in accordance with the respective standards can be removed from the washing chamber 70, that is to say items can be removed from the washing chamber in a hygienically cleaned state.

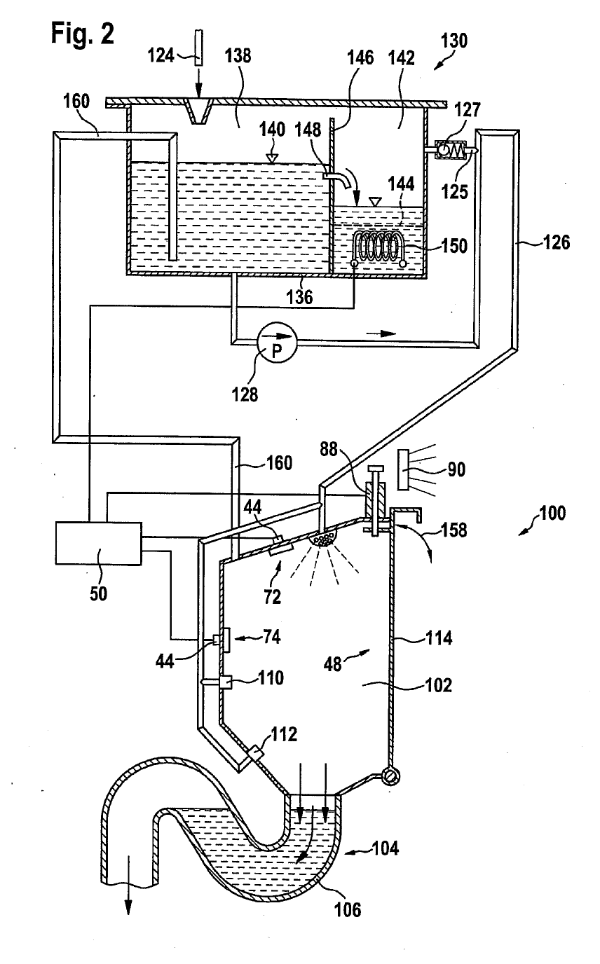

FIG. 2 shows a schematic illustration of a cleaning and disinfecting machine in which the method proposed according to the invention for assessing and monitoring the hygiene effect on items being cleaned can likewise be implemented.

FIG. 2 shows a cleaning and disinfecting machine 100 as used in a hospital or care homes for cleaning bedpans, urine bottles and also other collection containers for human excreta. Disinfection within the cleaning and disinfecting machine 100 can be carried out both thermally, that is to say using steam, and also chemically. To this end, a cleaning chamber 102 is generally formed in the cleaning and disinfecting machine 100, it being possible to load said cleaning chamber with containers to be cleaned from the outside via a door 114 which can be locked by means of the locking device 88 and through which the cleaned and disinfected containers can also be removed from the cleaning chamber 102 again after cleaning and disinfection is complete.

At its lower end, the cleaning chamber 102 comprises an outflow 104 which contains a siphon bend 106 and via which the remains of human excreta can be passed to a wastewater system. In order to avoid the formation of odors in the cleaning chamber 102, the siphon bend 106 which is formed in the outflow 104 and is generally provided as an odor barrier in wastewater systems on account of the water which collects in it, also serves the same purpose in the cleaning and disinfecting machine 100 described here. The cleaning chamber 102 of the cleaning and disinfecting machine 100 is accessible via a pivoting door 114. The door 114 can be moved about a hinge which is fitted at the lower end of the door 114 and can move in the opening/closing direction 158 in accordance with the double-headed arrow shown in the drawing. The cleaning chamber 102 can be subjected to steam by means of a water/steam unit 130. FIG. 2 illustrates nozzles 108, 110, 112 from which steam can enter the cleaning chamber 102. These nozzles are integrated in the roof surface of the cleaning chamber 102, but can also be provided in its lateral boundary surfaces. The nozzles 108, 110, 112 via which steam can be introduced into the cleaning chamber 102 could equally well be mounted on the rear boundary wall of the cleaning chamber 102. A safety overflow 160 furthermore issues into the cleaning chamber 102, via which safety overflow water can overflow from the water/steam unit 130 in the upper region of the cleaning and disinfecting machine 100 into the cleaning chamber 102 and pass from there into the outflow 104. The safety overflow 160 could also issue directly into the outflow 104.

The water/steam unit 130 is accommodated in the upper region of the cleaning and disinfecting machine 100 according to the illustration in FIG. 2. The water/steam unit 130 is assigned a water pump 128 which is arranged beneath the base 136 of the water/steam unit 130. The water pump 128, which increases the water pressure, is used to pump water out of a water container 138 of the water/steam unit 130 and into the cleaning chamber 102 via a water inflow 126. The water container 138 of the water/steam unit 130 is supplied via a cold-water inflow or a hot-water inflow 124. Depending on how the device is connected to the building, either cold water in a temperature range of between 10° C. and 30° C. flows into the water container 138 via the water inflow 124 or, if a hot-water connection is provided to the building, water with a temperature of between 45° C. and 60° C. can flow into the water container 138 of the water/steam unit 130. The water container 138 can also be filled with a mixture of hot and cold water. The hot water or cold water flowing in via the water inflow 124 flows into the water/steam unit 130 and fills it to a level which is identified by reference symbol 140. In the case of excess admission of water, water flows into the cleaning chamber 102 via a safety overflow 160 and is discharged into the wastewater system via the outflow 104 which is always open, with the result that no water damage can occur in the room in which the cleaning and disinfecting machine 100 is located.

The water container 138 is separated from a steam generator 142 of the water/steam unit 130 by a partition wall 146. In order to fill the steam generator 142 with water, an overflow line 148 extends through the partition wall 146. Water flows into the steam generator 142 via the overflow line 148. After water has been pumped away, a level 144 is established in the steam generator 142. In order to heat the water present in the steam generator 142, the water reservoir contained in the steam generator 142 is heated by means of a heating device 150 which is indicated in the drawing by a coil. The steam which is produced as the water is heated is conveyed into the feed line 126 which leads from the water pump 128 to the cleaning chamber 102 via a line section 125. The line section 125 between the steam generator 142 and the feed line 126 to the cleaning chamber 102 is closed by a non-return valve 127. The non-return valve 127 opens on account of the pressure of the steam in the steam generator 142, with the result that steam can flow into the feed line 126 to the cleaning chamber 102. By virtue of the water pump 128, water from the water container 138 can also be sprayed into the cleaning chamber 102 via the nozzles 108 on the ceiling of the cleaning chamber 102 and/or via the nozzles 110, 112 which are arranged in the rear region of the cleaning chamber 102. The nozzles 108, 110, 112 can also be arranged on the side walls or on that face of the appliance door 114, which closes the cleaning chamber 102, which faces the cleaning chamber 102.

The program run within the cleaning and disinfecting machine 100 can be controlled by means of a control system 50 which is constructed in an analogous manner to the control system 50 of the single-chamber dishwasher 10. The heat equivalence values in accordance with the NSF 3 standard or in accordance with EN ISO 15883 are implemented in the control system 50. The temperature sensor 44 which is permanently installed in the cleaning chamber 102 in the installation positions 72 and 74 illustrated by way of example detects the respectively established temperature during the program run within the cleaning and disinfecting machine 100. At the same time, the control system 50 determines the action time at the respective temperature. The control system 50 calculates the corresponding heat equivalence values from this information. The calculated heat equivalence values are then compared with values for heat equivalents in accordance with the NSF standard 3 or EN ISO 15883 which are stored in the control system 50, in order to assess the hygiene effect of the cleaning and disinfecting machine 100 which is respectively established in the cleaning chamber 102. The heat equivalence values which are established during operation of the cleaning and disinfecting machine 100 can be continuously indicated to the user or to the operator of the cleaning and disinfecting machine 100 by means of a display which is arranged on the cleaning and disinfecting machine 100. Analogously to the illustration according to FIG. 1, the locking mechanism 88, which is assigned to the appliance door 114 and which can be designed as an electromagnet, can be unlocked by means of the control system 50, which is assigned to the cleaning and disinfecting machine, when the heat equivalence values which ensure an adequate hygiene effect are reached. An acoustic or an optical signal can also be used to indicate that the prescribed heat equivalents have been reached within the cleaning chamber 102 of the cleaning and disinfecting machine 100.

After the prescribed heat equivalent values are reached within the cleaning chamber 102 of the cleaning and disinfecting machine 100, hygienically cleaned items can be readily removed from the cleaning chamber 102.

### LIST OF REFERENCE SYMBOLS

10 Single-chamber dishwasher
12 Rack
14. Items to be washed
16 Tank for washing liquor
18 Heating element for washing liquor
20 Circulation pump for washing liquor
22 Washing system for washing liquor
24 Nozzles for washing liquor
26 Fresh-water boiler
28 Filling valve
30 Fresh water
32 Heating element for fresh-water tank
34 Cover level
36 Ventilation line
38 Suction point
40 Washing system for fresh water
42 Nozzles for fresh water
44 Temperature sensor
46 Appliance door
48 Loading opening
50 Control system
52 Microprocessor (CPU)
54 Data memory
56 Main control line
58 Measurement-data detection unit
60 Power controller for circulation pump 20
62 Electrical power supply for circulation pump 20
64 Fresh-water pump
66 Electrical power supply for fresh-water pump
68 Power controller for fresh-water pump 64
70 Washing chamber
72 First temperature-sensor position
74 Second temperature-sensor position
76 Fresh-water line
78 Washing-liquor line
80 Power supply for heating element 18
82 Power controller for heating element
84 Power supply for heating element 32
86 Power controller for heating element 32
88 Locking device
90 Display
100 Cleaning and disinfecting machine
102 Cleaning chamber
104 Outflow
106 Siphon bend
108 First nozzle
110 Second nozzle
112 Third nozzle
114 Appliance door
124 Cold-water/hot-water inflow
125 Line section
126 Feed line for cleaning chamber
127 Non-return valve
128 Water pump
130 Water/steam unit
136 Base
138 Water container
140 Water level in the washing container
142 Steam generator
144 Water level in the steam generator
146 Partition wall
148 Overflow line
150 Heating device
158 Opening/closing direction
160 Safety overflow

1. A method for assessing and guaranteeing the thermal hygiene effect on items to be washed which are accommodated in a washing chamber of a dishwasher or on items to be cleaned which are accommodated in a cleaning chamber of a cleaning and disinfecting machine, with a temperature sensor being permanently installed in the washing chamber or in the cleaning chamber, characterized in that the thermal hygiene effect the temperature and action time acting on the items to be washed or on the items to be cleaned are detected by means of the temperature sensor and a control system, and corresponding heat equivalent values are calculated from this information in order to assess the thermal hygiene effect.

2. The method as claimed in claim 1, wherein the thermal hygiene effect is assessed by the control system on the basis of the NSF standard 3 regulation whose values for heat equivalents HUE are stored in a data memory of the control system.

3. The method as claimed in claim 1, wherein the thermal hygiene effect is assessed by the control system of the single-chamber dishwasher or the cleaning and disinfecting machine in accordance with the A₀ values of EN ISO 15883, annex A.

4. The method as claimed in claim 1, wherein a program step which runs within the washing chamber or within the cleaning chamber in order to guarantee the thermal hygiene effect is terminated after a preset value of heat equivalents, which has to be reached at the minimum, according to NSF 3 standard or EN ISP 15883 is reached.

5. The method as claimed in claim 4, wherein the end of the program step as claimed in claim 4 is indicated by a visual signal.

6. The method as claimed in claim 4, wherein the end of the program step as claimed in claim 4 is indicated by an acoustic signal.

7. The method as claimed in claim 4, wherein an appliance door to the washing chamber of the single-chamber dishwashing machine or to the cleaning chamber of the cleaning and disinfecting machine is locked when a program run starts and is released again after a value for the heat equivalents which is preset in the control system is reached.

8. The method as claimed in claim 1, wherein the value for the heat equivalents which is actually reached in each case is indicated to the user by means of a visual display during the program run within the washing chamber or the cleaning chamber.

9. The method as claimed in claim 1, wherein the A₀ value which is actually reached in each case is indicated to the user of the single-chamber dishwashing machine or the cleaning and disinfecting machine by means of an acoustic signal during the program run within the washing chamber or the cleaning chamber.

10. A single-chamber dishwashing machine for carrying out the method according to claim 1, wherein the temperature sensor is permanently installed in a first temperature-sensor position or a second temperature-sensor position in the washing chamber of the single-chamber dishwashing machine.

11. The single-chamber dishwashing machine as claimed in claim 10, wherein the temperature sensor is accommodated in the upper region of the washing chamber.

12. The single-chamber dishwashing machine as claimed in claim 10, wherein the temperature sensor is arranged opposite the appliance door, on the rear face of the washing chamber.

13. A cleaning and disinfecting machine for carrying out the method as claimed in claim 1, wherein the temperature sensor is permanently installed in a first temperature-sensor position or a second temperature-sensor position in the cleaning chamber of the cleaning and disinfecting machine.

14. The cleaning and disinfecting machine as claimed in claim 13, wherein the temperature sensor is accommodated in the upper region of the cleaning chamber.

15. The cleaning and disinfecting machine as claimed in claim 13, wherein the temperature sensor is arranged opposite the appliance door, on the rear face of the cleaning chamber.
